(19) United States
(12) Patent Application Publication
(10) Pub. No.: US 2013/0165471 A1
(43) Pub. Date: Jun. 27, 2013

(54) 2-SUBSTITUTED-THIENOQUINOLONES AND RELATED COMPOUNDS AS ANTI-INFECTIVE AGENTS

(71) Applicants: Avinash Phadke, Branford, CT (US); Jason Allan Wiles, Madison, CT (US)

(72) Inventors: Avinash Phadke, Branford, CT (US); Jason Allan Wiles, Madison, CT (US)

(73) Assignee: Achillion Pharmaceuticals Inc., New Haven, CT (US)

(21) Appl. No.: 13/723,860

(22) Filed: Dec. 21, 2012

Related U.S. Application Data
(60) Provisional application No. 61/578,412, filed on Dec. 21, 2011.

(51) Int. Cl. C07D 495/04

(52) U.S. Cl. CPC C07D 495/04
USPC 514/291; 546/80

(57) ABSTRACT

Disclosed herein are 2-substituted-thienoquinolones and related compounds and their pharmaceutically acceptable salts useful as antiviral agents and having the general formula

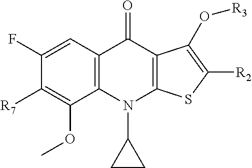

in which the variables R₂, R₃, and R₇ are defined herein. Certain compounds provided herein possess potent antibacterial, antiprotozoal, or antifungal activity and are particularly efficacious for the treatment of MRSA infections. The invention also provides pharmaceutical compositions, pharmaceutical compositions containing a 2-substituted-thienoquinolone in combination with one or more other active agent, and methods of treating microbial infections in animals by administering an effective amount of a 2-substituted-thienoquinolone to an animal suffering from a microbial infection.

### CROSS REFERENCE TO RELATED APPLICATION

This application claims priority from U.S. Provisional application No. 61/578,412, filed Dec. 21, 2011, which is hereby incorporated by reference in its entirety.

### FIELD OF THE INVENTION

Disclosed herein are 2-substituted-thienoquinolones and related compounds useful as antimicrobial agents. Certain compounds provided herein possess potent antibacterial, antiprotozoal, or antifungal activity. Particular compounds provided herein are also potent and/or selective inhibitors of prokaryotic DNA synthesis and prokaryotic reproduction. The invention also provides anti-microbial compositions, including pharmaceutical compositions, containing one or more carrier, diluents, and/or excipients. The invention provides pharmaceutical compositions containing a hydroxylthienoquinolone or related compound as the only active agent or containing a hydroxylthienoquinolone or related compound in combination with one or more other active agent, such as one or more other antimicrobial or antifungal agent. The invention further provides methods for treating microbial infections in animals by administering an effective amount of a hydroxylthienoquinolone or related compound to an animal suffering from a microbial infection. The invention also provides methods of inhibiting microbial growth and survival by applying an effective amount of a 2-substituted-thienoquinolones or related compound.

### BACKGROUND OF THE INVENTION

Antimicrobial compounds are compounds capable of destroying or suppressing the growth or reproduction of microorganisms, such as bacteria, protozoa, mycoplasma, yeast, and fungi. The mechanisms by which antimicrobial compounds act vary. However, they are generally believed to function in one or more of the following ways: by inhibiting cell wall synthesis or repair; by altering cell wall permeability; by inhibiting protein synthesis; or by inhibiting synthesis of nucleic acids. For example, beta-lactam antibacterials inhibit the essential penicillin binding proteins (PBPs) in bacteria, which are responsible for cell wall synthesis. Quinolones act, at least in part, by inhibiting synthesis of DNA, thus preventing the cell from replicating.

Many attempts to produce improved antimicrobials yield equivocal results. Indeed, few antimicrobials are produced that are truly clinically acceptable in terms of their spectrum of antimicrobial activity, avoidance of microbial resistance, and pharmacology. There is a continuing need for broad-spectrum antimicrobials, and a particular need for antimicrobials effective against resistant microbes.

Pathogenic bacteria are known to acquire resistance via several distinct mechanisms including inactivation of the antibiotic by bacterial enzymes (e.g., beta-lactamases that hydrolyze penicillin and cephalosporins); removal of the antibiotic using efflux pumps; modification of the target of the antibiotic via mutation and genetic recombination (e.g., penicillin-resistance in Neiserria gonorrhea); and acquisition of a readily transferable gene from an external source to create a resistant target (e.g., methicillin-resistance in Staphylococcus aureus). There are certain Gram-positive pathogens, such as vancomycin-resistant Enterococcus faecium, which are resistant to virtually all commercially available antibiotics.

Resistant organisms of particular note include methicillin-resistant and vancomycin-resistant Staphylococcus aureus, penicillin-resistant Streptococcus pneumoniae, vancomycin-resistant enterococci, fluoroquinolone-resistant E. coli, cephalosporin-resistant aerobic gram-negative rods and imipenem-resistant Pseudomonas aeruginosa. These organisms are significant causes of nosocomial infections and are clearly associated with increasing morbidity and mortality. The increasing numbers of elderly and immunocompromised patients are particularly at risk for infection with these pathogens. Therefore, there is a large unmet medical need for the development of new antimicrobial agents. In recent years Methicillin Resistant Staphylococcus Aureus (MRSA) infections have become more common, particularly in institutional and hospital settings. Up to 60% of staphylococcus infections are attributable to methicillin resistant strains in some parts of the United States. Some MRSA strains are now resistant to both Vancomycin and Gentamicin, drugs once considered the last defense against staphylococcus infections. Thus, there is a particularly urgent need for drugs effective against MRSA strains.

Quinoline compounds effective against methicillin susceptible Staphylococcus aureus have been previously identified. For example, see Japanese laid-open publication no. 03-223289. JP 03-223289 discusses compounds having activity against MSSA rather than MRSA. Generally quinolone and isothiazoloquinolone compounds are 32-128 fold less active against MRSA than MSSA. Thus one of skill in the art would not expect compounds discussed in JP 03-223289 to be effective against MRSA. The inventors have unexpectedly identified a class of 2-substituted-thienoquinolones that have an unexpectedly high MRSA: MSSA EC50 ratio, as measured by MIC assay, and thus quite useful for treating MRSA infections. The unexplained improved MSSA/MRSA MIC ratio also suggests an alternate mode of action or binding at the active site for the newly discovered compounds.

The present invention fulfills the need for drugs effective against MRSA, and provides further related advantages.

### SUMMARY

This disclosure provides 2-substituted-thienoquinolone compounds of Formula I (shown below). The disclosure provides compounds of Formula I, which possess potent and/or selective antibacterial, antiprotozoal, or antifungal activity. The disclosure also provides pharmaceutical compositions containing one or more compounds of Formula I or related compounds together with one or more pharmaceutically acceptable carriers.

The disclosure further provides methods of treating microbial infections, particularly bacterial and protozoal infections by administering an effective amount of a compound of Formula I or related compound to a patient suffering from a microbial infection. These microbial infections include bacterial infections, for example E. coli infections, Staphylococcus infections, particularly including Methicillin Resistant Staphylococcus Aureus infections, Salmonella infections and protozoal infections, for example Chlamydia infections. The disclosure particularly includes methods of treating microbial infections in mammals, including human patients, but also encompasses methods of treating microbial infections in other animals, including fish, birds, reptiles, and amphibians.

Methods of treatment include administering a compound of Formula I as the single active agent or administering a compound of Formula I in combination with one or more other therapeutic agent, such as an antibacterial, an antifungal, an antiviral, an interferon or other immune system modulator, an efflux-pump inhibitor, a beta-lactamase inhibitor, an anti-inflammatory, or another compound of Formula I.

Thus, compounds of Formula I

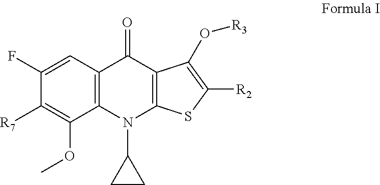

and the pharmaceutically acceptable salts thereof, are provided herein. Within Formula I, the variables, e.g. R₂, R₃, and R₇, carry the definitions set forth below.

R₂ is amino, halogen, or —CORₐ, where Rₐ is C₁-C₆alkyl.

R₃ is hydrogen, C₁-C₆alkyl, C₁-C₆alkanoyl, (C₁C₆alkylester)C₁-C₄alkyl, or a benzenesulfonyl group, each of which R₃ other than hydrogen is optionally substituted.

R₇ is a nitrogen-linked 5- to 6-membered heterocycloalkyl group, having 0 or 1 additional ring heteroatoms selected from N, O, and S, which nitrogen-linked 5- to 6-membered heterocycloalkyl group is substituted with at least 1 substituent independently chosen from (a) or (b) and 0 or 1 or more substituents independently chosen from (c); wherein
(a) is amino, aminoC₁-C₂alkyl, and aminoC₁-C₃alkenyl;
(b) is (C₁-C₆alkylcarbamate)C₀-C₄alkyl, (C₁-C₆alkylcarboxamide)C₀-C₄alkyl, and (benzenesulfonylamino)C₀-C₄alkyl, where each of (a) and (b) other than amino is substituted with 0 to 2 substituents independently chosen from halogen, amino, C₁-C₄alkyl, C₁-C₄alkoxy; and
(c) is halogen, hydroxyl, amino, C₁-C₄alkyl, C₂-C₄alkenyl, C₁-C₄alkoxy, C₁-C₂haloalkyl, and C₁-C₂haloalkoxy.

### DETAILED DESCRIPTION OF THE INVENTION

### Chemical Description and Terminology

In this disclosure compounds are generally described using standard nomenclature.

In certain situations, the compounds of Formula I may contain one or more asymmetric elements such as stereogenic centers, stereogenic axes and the like, e.g. asymmetric carbon atoms, so that the compounds can exist in different stereoisomeric forms. “Formula I” includes compounds and salts of certain subformulae, described herein such as compounds of Formula II to V as well as related compounds of Formula A, B, and C. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms, with all isomeric forms of the compounds being included in the present disclosure. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

Where a compound exists in various tautomeric forms, the disclosure is not limited to any one of the specific tautomers, but rather includes all tautomeric forms.

The present disclosure is includes all isotopes of atoms occurring in the present compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Certain compounds are described herein using a general formula that includes variables, e.g. A₁, R₂, R₃, R₅, A₆, R₇, A₈, and R₉. Unless otherwise specified, each variable within such a formula is defined independently of other variables. Thus, if a group is said to be substituted, e.g. with 0-2 R*, then said group may be substituted with up to two R* groups and R* at each occurrence is selected independently from the definition of R*. Also, combinations of substituents and/or variables are permissible only if such combinations result in stable compounds. When a group is substituted by an “oxo” substituent a carbonyl bond replaces two hydrogen atoms on a carbon. An “oxo” substituent on an aromatic group or heteroaromatic group destroys the aromatic character of that group, e.g. a pyridyl substituted with oxo is a pyridone.

The term “substituted,” as used herein, means that any one or more hydrogen atoms bound to the designated atom or group is replaced with a selection from the indicated group, provided that the designated atom's normal valence is not exceeded. When a substituent is oxo (i.e., ═O), then 2 hydrogen atoms on the substituted atom are replaced with a double-bonded oxygen. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. Unless otherwise specified substituents are named into the core structure. For example, it is to be understood that when (cycloalkyl)alkyl is listed as a possible substituent the point of attachment of this substituent to the core structure is in the alkyl portion.

Substituents are named into the ring unless otherwise indicated. A dash (“-”) or a double bond (“=”) that is not between two letters or symbols indicates the point of attachment for a substituent. For example, —CONH₂ is attached through the carbon atom.

As used herein, “alkyl” is includes both branched and straight-chain saturated aliphatic hydrocarbon groups, having the specified number of carbon atoms. Thus, the term C₁-C₆alkyl as used herein includes alkyl groups having from 1 to about 6 carbon atoms. When C₀-Cₙ alkyl is used herein in conjunction with another group, for example, (aryl)C₀-C₄ alkyl, the indicated group, in this case aryl, is either directly bound by a single covalent bond (C₀), or attached by an alkyl chain having the specified number of carbon atoms, in this case from 1 to about 4 carbon atoms. Examples of alkyl include, but are not limited to, methyl, ethyl, n-propyl, isopropyl, n-butyl, t-butyl, n-pentyl, and sec-pentyl. C₁-C₆alkyl includes alkyl groups have 1, 2, 3, 4, 5, or 6 carbon atoms.

“Alkenyl” as used herein, indicates a hydrocarbon chain of either a straight or branched configuration having one or more carbon-carbon double bond bonds, which may occur at any stable point along the chain. Examples of alkenyl groups include ethenyl and propenyl. “Aminoalkenyl” is an alkenyl group having a terminal amino substituent. When an alkenyl (or aminoalkenyl) substitutes a cycloalkyl or heterocycloalkyl group the carbon-carbon double bound may be between a ring carbon of the cyclic group and the first carbon of the alkenyl substituent.

“Alkanoyl indicates an alkyl group as defined above, attached through a keto —(C═O)— bridge. Alkanoyl groups have the indicated number of carbon atoms, with the carbon of the keto group being included in the numbered carbon atoms. For example a C₂alkanoyl group is an acetyl group having the formula CH₃(C═O)—.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Aminoalkyl” indicates an amino group attached through an alkyl group, wherein the alkyl group is as defined above and has the indicated number of carbon atoms.

“Alkylcarbamate” indicates a group having the formula alkyl-O—(C═O)—NH—, wherein the alkyl group is as defined above and has the indicated number of carbon atoms. An “(alkylcarbamate)alkyl”, is an alkylcarbamate as defined that is directly bound to the group it substitutes (C₀alkyl) or attached to the group it substitutes through an alkyl linker having the specified number of carbon atoms.

Alkylcarboxamide” indicates a group having the formula alkyl-(C═O)—NH—, wherein the alkyl group is as defined above and has the indicated number of carbon atoms. An “(alkylcarboxamide)alkyl”, is an alkylcarboxamide as defined that is directly bound to the group it substitutes (C₀alkyl) or attached to the group it substitutes through an alkyl linker having the specified number of carbon atoms.

“Alkylester” indicates a group having the formula alkyl-O—(C═O)— or alkyl-(C═O)—O—, wherein the alkyl group is as defined above and has the indicated number of carbon atoms. An “(alkylester)alkyl”, is an alkylester as defined that is attached to the group it substitutes through an alkyl linker having the specified number of carbon atoms.

“Aryl” indicates aromatic groups containing only carbon in the aromatic ring or rings. Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Such substitution may include fusion to a 5 to 7-membered saturated cyclic group that optionally contains 1 or 2 heteroatoms independently chosen from N, O, and S, to form, for example, a 3,4-methylenedioxy-phenyl group. Aryl groups include, for example, phenyl, naphthyl, including 1-naphthyl and 2-naphthyl, and bi-phenyl. “Aryloxy” is an aryl groups as defined above, attached to the group it substitutes via a covalently bound oxygen atom.

“Cycloalkyl” as used herein, indicates saturated hydrocarbon ring groups, having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norborane or adamantane. In the term “(cyclolalkyl)alkyl,” cycloalkyl and alkyl are as defined above, and the point of attachment in on the alkyl group. In the term “(cyclolalkyl)aminoC₀-C₀alkyl,” cycloalkyl, amino, and alkyl are as defined above, and the point of attachment in on the alkyl group or through a nitrogen bridge (C₀alkyl).

“Cycloalkenyl” as used herein, indicates an unsaturated, but not aromatic, hydrocarbon ring having at least one carbon-carbon double bond. Cycloalkenyl groups contain from 4 to about 8 carbon atoms, usually from 4 to about 7 carbon atoms. Examples include cyclohexenyl and cyclobutenyl.

“Haloalkyl” indicates both branched and straight-chain saturated aliphatic hydrocarbon groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoro ethyl.

“Haloalkoxy” indicates a haloalkyl group as defined above attached through an oxygen bridge.

“Halo” or “halogen” as used herein refers to fluoro, chloro, bromo, or iodo.

The term “heterocycloalkyl” indicates a saturated cyclic group containing from 1 to about 3 heteroatoms chosen from N, O, and S, with remaining ring atoms being carbon. Heterocycloalkyl groups have from 3 to about 8 ring atoms, and more typically have from 5 to 7 ring atoms. Examples of heterocycloalkyl groups include morpholinyl, piperazinyl, piperidinyl, and pyrrolidinyl groups.

The term “heterocyclic group” indicates a monocyclic saturated, partially unsaturated, or aromatic ring containing from 1 to about 4 heteroatoms chosen from N, O, and S, with remaining ring atoms being carbon, or a bicyclic saturated, partially unsaturated, or aromatic heterocyclic ring system containing at least 1 heteroatom in the two ring system chosen from N, O, and S and containing up to about 4 heteroatoms independently chosen from N, O, and S in each ring of the two ring system. Usually each ring of the heterocyclic group contains from 4-6 ring atoms but some other number of ring atoms may be specified. Unless otherwise indicated, the heterocyclic ring may be attached to its pendant group at any heteroatom or carbon atom that results in a stable structure. When indicated the heterocyclic rings described herein may be substituted on carbon or on a nitrogen atom if the resulting compound is stable. It is preferred that the total number of heteroatoms in a heterocyclic groups is not more than 4 and that the total number of S and O atoms in a heterocyclic group is not more than 2, more preferably not more than 1. Examples of heterocyclic groups include, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, dihydroisoindolyl, 5,6,7,8-tetrahydroisoquinoline, pyridinyl, pyrimidinyl, furanyl, thienyl, pyrrolyl, pyrazolyl, pyrrolidinyl, morpholinyl, piperazinyl, piperidinyl, and pyrrolidinyl.

Additional examples heterocyclic groups include, but are not limited to, phthalazinyl, oxazolyl, indolizinyl, indazolyl, benzothiazolyl, benzimidazolyl, benzofuranyl, benzoisoxolyl, dihydro-benzodioxinyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanonyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, 5 pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromanyl, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N oxide, indazolyl N-oxide, benzothiazolyl N-oxide, benzimidazolyl N-oxide, pyrrolyl N-oxide, oxadiazolyl N-oxide, thiadiazolyl N-oxide, tetrazolyl N-oxide, benzothiopyranyl S-oxide, and benzothiopyranyl S,S-dioxide.

As used herein “active agents” are compounds that have pharmaceutical utility, e.g. may be used to treat a patient suffering from a disease or condition, or may be used prophylactically to prevent the onset of a disease or condition in a patient, or that may be used to enhance the pharmaceutical activity of other compounds.

“Pharmaceutical compositions” are compositions comprising at least one active agent, such as a compound or salt of Formula I and at least one other excipient. “Carriers” are any inactive materials, including excipients and diluents, that may be added to the pharmaceutical compositions including carriers and diluents. Pharmaceutical compositions meet the U.S. FDA's GMP (good manufacturing practice) standards for human or non-human drugs.

“Salts” of the compounds of the present invention include inorganic and organic acid and base addition salts. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

“Pharmaceutically acceptable salts” includes derivatives of the disclosed compounds wherein the parent compound is modified by making non-toxic acid or base salts thereof, and further refers to pharmaceutically acceptable hydrates solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxylmaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

The term “therapeutically effective amount” of a compound of Formula I, or a related formula, means an amount effective, when administered to a human or non-human patient, to provide a therapeutic benefit such as an amelioration of symptoms, e.g., an amount effective to decrease the symptoms of a microbial infection, and including an amount sufficient to reduce the symptoms of a bacterial, fungal, or protozoal infection. In certain circumstances a patient suffering from a microbial infection may not present symptoms of being infected. Thus a therapeutically effective amount of a compound is also an amount sufficient significantly reduce the detectable level of microorganism or antibodies against the microorganism in the patient's blood, serum, other bodily fluids, or tissues. The invention also includes, in certain embodiments, using compounds of Formula I in prophylactic treatment and therapeutic treatment. In the context of prophylactic or preventative treatment a “therapeutically effective amount” is an amount sufficient to significantly decrease the treated patient's risk of contracting a microorganism infection. For example, prophylactic treatment may be administered when a subject will knowingly be exposed to infectious microbes. A significant reduction is any detectable negative change that is statistically significant in a standard parametric test of statistical significance such as Student's T-test, where p<0.05.

### Antimicrobial Compounds

In addition to the compounds of Formula I described above, the invention also includes the following embodiments.

The invention includes compounds and salts of the Formula I

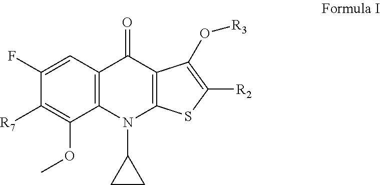

as described in the Summary section, wherein the compound is not: 9-cyclopropyl-6-fluoro-3-hydroxyl-8-methoxy-7-(piperazin-1-yl)-2-pivaloylthieno[2,3-b]quinolin-4(9H)-one; 2-acetyl-9-cyclopropyl-6-fluoro-3-hydroxyl-8-methoxy-7-(piperazin-1-yl)thieno[2,3-b]quinolin-4(9H)-one; (R)-2-acetyl-7-(3-(2-aminopropan-2-yl)pyrrolidin-1-yl)-9-cyclopropyl-6-fluoro-3-hydroxyl-8-methoxythieno[2,3-b]quinolin-4(9H)-one; (S)-2-acetyl-7-(3-(aminomethyl)pyrrolidin-1-yl)-9-cyclopropyl-6-fluoro-3-hydroxyl-8-methoxythieno[2,3-b]quinolin-4(9H)-one; (R)-7-(3-(2-aminopropan-2-yl)pyrrolidin-1-yl)-9-cyclopropyl-6-fluoro-3-hydroxyl-8-methoxy-2-propionylthieno[2,3-b]quinolin-4(9H)-one; 2-acetyl-7-(3-(aminomethyl)piperidin-1-yl)-9-cyclopropyl-6-fluoro-3-hydroxyl-8-methoxythieno[2,3-b]quinolin-4(9H)-one; or 2-acetyl-7-(3-(2-amino-1-fluoroethylidene)piperidin-1-yl)-9-cyclopropyl-6-fluoro-3-hydroxyl-8-methoxythieno[2,3-b]quinolin-4(9H)-one.

Included herein are compounds of the Formula I in keto and enol form as shown below. All possible tautomers are included.

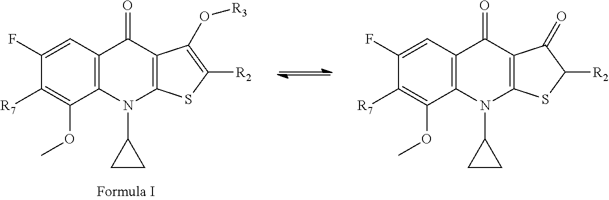

The invention also includes compounds of Formula I in which certain variables carry the definitions set forth below. Variables for which a definition is not specified carry the definition set forth for compounds of Formula I in the Summary of Invention section. Compounds may carry any combination of the variable definitions disclosed herein, so long as a stable compound results.

### The R₂ Variable

R₂ may carry any of the following values.

R₂ is amino, halogen, or —CORₐ, where Rₐ is C₁-C₆alkyl.

R₂ is —CORₐ, where Rₐ is C₁-C₆alkyl.

R₂ is acetyl.

### The R₃ Variable

R₃ may carry any of the following definitions.

R₃ is hydrogen or an optionally substituted benzenesulfonyl group.

R₃ is hydrogen, or a benzenesulfonyl group substituted with 0 or 1 or more substituents independently chosen from halogen, hydroxyl, amino, C₁-C₄alkyl, C₂-C₄alkenyl, C₁-C₄alkoxy, C₁-C₂haloalkyl, and C₁-C₂haloalkoxy.

R₃ is hydrogen.

R₃ is a benzenesulfonyl group substituted with 0 or 1 or 2 substituents independently chosen from halogen, hydroxyl, amino, methyl, methoxy, trifluoromethyl, and trifluoromethoxy.

R₃ is C₁-C₄alkanoyl or R₃ is (C₁-C₆alkylester)C₁-C₂alkyl.

### The R₇ Variable

R₇ may carry any of the following definitions.

R₇ is a nitrogen-linked 5- to 6-membered heterocycloalkyl group, having 0 or 1 additional ring heteroatoms selected from N, O, and S, which nitrogen-linked 5- to 6-membered heterocycloalkyl group is substituted with at least 1 substituent independently chosen from (a) or (b) and 0 or 1 or more substituents independently chosen from (c); wherein
(a) is amino, aminoC₁-C₂alkyl, and aminoC₁-C₃alkenyl;
(b) is (C₁-C₆alkylcarbamate)C₀-C₄alkyl, (C₁-C₆alkylcarboxamide)C₀-C₄alkyl, and (benzenesulfonylamino)C₀-C₄alkyl, where each of (a) and (b) other than amino is substituted with 0 to 2 substituents independently chosen from halogen, amino, and C₁-C₄alkyl; and
(c) is halogen, hydroxyl, amino, C₁-C₄alkyl, C₂-C₄alkenyl, C₁-C₄alkoxy, C₁-C₂haloalkyl, and C₁-C₂haloalkoxy.

R₇ is pyrrolidinyl or piperidinyl, each of which is substituted with at least 1 substituent independently chosen from (a) or (b) and 0 or 1 or 2 substituents chosen from (c).

R₇ is pyrrolidinyl or piperidinyl, each of which is substituted with at least 1 substituent independently chosen from (a) or (b); wherein (a) is amino and aminoC₁-C₂alkyl; and (b) is (C₁-C₆alkylcarbamate)C₁-C₂alkyl, (C₁-C₂alkylcarboxamide)C₁-C₂alkyl, and (benzenesulfonylamino)C₁-C₂alkyl, where each of (b) is substituted with 0 to 2 substituents independently chosen from halogen, amino, and methyl.

Any of the above variable definitions may be combined for a compound of Formula I or any of the subformulae thereof, so long as a stable compound results.

Compounds having improved cytotoxicity are provided herein. For example, compounds in which R₂ is acetyl exhibit surprisingly low cellular toxicity compared to other compound of Formula I and other structurally similar compounds. For example certain compounds provided herein exhibit surprisingly low cytotoxicity in the assay of Example 4.

Compounds, which are potent inhibitors of MRSA are provided herein. For example certain compounds provided herein are potent inhibitors of S. aureaus strains 700699, BSA643, and BSA648, NY2746.

Compounds that have an unexpectedly high MRSA: MSSA EC50 ratio, as measured by MIC assay, are provided herein. Typically a compound's MSSA activity is an order of magnitude or more than the compound's MRSA activity. For example an MRSA:MSSA activity ratio of 1:10 or 1:50 is typical, in contrast with the 1:4 to 1:8 activity ratio range observed with the compounds herein. Thus the compounds disclosed herein are particularly useful for treating MRSA infections. The unexpectedly improved MSSA/MRSA MIC ratio also suggests an alternate mode of action or binding at the active site for the newly discovered compounds.

### Anti-Microbial and Pharmaceutical Preparations

The invention provides pharmaceutical compositions, comprising a compound or salt thereof of Formula I, together with at least one pharmaceutically acceptable excipient.

Compounds described herein can be administered as the neat chemical, but are specifically administered as a pharmaceutical composition, for example compounds can be administered a pharmaceutical formulation compound of Formula I or related compound of Formula II to V or a or pharmaceutically acceptable salt thereof, together with at least one pharmaceutically acceptable carrier.

The compounds described herein may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, an injectable fluid, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorings, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active and/or inactive agents may be included in the pharmaceutical compositions, provided that such agents do not substantially interfere with the activity of the Aryl substituted thiazol-2-yl-piperidines and related compounds used in the pharmaceutical compositions. The optional active is an additional active agent that is not a compound or salt of Formula I.

The pharmaceutical compositions can be formulated for oral administration. These compositions contain between 0.1 and 99 weight % (wt. %) of a compound of Formula I or related compound or salt and usually at least about 5 wt. % of a compound or salt of Formula I Some embodiments contain from about 25 wt. % to about 50 wt. % or from about 5 wt. % to about 75 wt. % of the compound or salt of Formula I or related compound.

Amount of compound or salt of Formula I in a unit dose may be generally varied or adjusted from about 1.0 milligram to about 1,000 milligrams, from about 1.0 to about 950 milligrams, from about 1.0 to about 500 milligrams, or from about 1 to about 250 milligrams, according to the particular application and the potency of the compound. The actual dosage employed may be varied depending upon the patient's age, sex, weight and severity of the condition being treated.

### Packaged Formulations

The invention includes packaged pharmaceutical formulations. Such packaged formulations include a pharmaceutical composition containing one or more compounds or salts of Formula I in a container and instructions for using the composition to treat an animal (typically a human patient) suffering from a microorganism infection, or prevent a microorganism infection in an animal.

The instructions may also be instructions for using the composition to treat a patient suffering from a bacterial infection, such as a S. aureus infection, including MRSA infection.

In all of the foregoing the compounds of the invention can be administered alone or as mixtures, and the compositions may further include additional drugs or excipients as appropriate for the indication.

### Methods of Treatment

The invention includes methods of treating microorganism infections, particularly bacterial and protozoal infections, by administering an effective amount of one or more compounds of Formula I to a patient at risk for a microorganism infection or suffering from a microorganism infection. Treatment of human patients is particularly contemplated. However, treatment of non-human patients is within the scope of the invention. The invention includes treatment or prevention of microbial infections in fish, amphibians, reptiles or birds, but is the invention includes treating mammals. Methods of treating microorganism infections in livestock animals, companion animals, and human patients are particularly preferred.

The compounds disclosed herein are useful for treating bacterial infections in animals. Furthermore compounds of the invention may be used to treat a variety of conditions not attributed to bacterial infections. These include diseases and disorders caused fungal infections, mycoplasma infections, protozoal infections, or other conditions involving infectious organisms.

In some circumstances an effective amount of a compound of Formula I may be an amount sufficient to reduce the symptoms of the microorganism infection. Alternatively an effective amount of a Compound of Formula I may be an amount sufficient to significantly reduce the amount of microorganism or antibodies against the detectable in a patient's tissues or bodily fluids.

Methods of treatment also include inhibiting microorganism replication in vivo, in an animal at risk for a microorganism infection or suffering from such an infection, by administering a sufficient concentration of a compound of Formula I to inhibit bacterial survival in vitro. By “sufficient concentration” of a compound administered to the patient is meant the concentration of the compound available in the animal's system to prevent or combat the infection. Such a concentration by be ascertained experimentally, for example by assaying blood concentration of the compound, or theoretically, by calculating bioavailability. The amount of a compound sufficient to inhibit bacterial survival in vitro may be determined with a conventional assay for bacterial survival such as the Minimum Inhibitory Concentration (MIC) Assay disclosed in Example 4, which follows.

The invention also includes using compounds of Formula I in prophylactic therapies. In the context of prophylactic or preventative treatment an effective amount of a compound of the invention is an amount sufficient to significantly decrease the treated animal's risk of contracting a microorganism infection.

Compounds of the invention are particularly useful for treating infectious disorders. These include for example: ocular infections such as conjunctivitis; urinary tract and genital infections, such as complicated urinary tract infections, acute urinary tract and genital infections, such as pyelonephritis, cervical gonococcal infections, cystitis, urethral chlamydial infections, cervical chlamydial infections, urethral gonococcal infections, and prostatitis, respiratory infections, such as lower respiratory tract infections, acute sinusitis, acute exacerbations of chronic bronchitis, community-acquired pneumonia, and nosocomial pneumonia, skin infections, such as skin-structure infections, impetigo, folliculitis, boils, scalded skin syndrome, and cellulites, and other infections such as bone infections, joint infections, infectious diarrhea, typhoid fever, intra-abdominal infections, gynecologic infections, including toxic shock syndrome, pelvic infections, and post-surgical infections. In a preferred embodiment, compounds of Formula I are used to treat Staphylococcus infections, especially Methicillan Resistant Staphylococcus aureus infections.

The disclosed compounds are useful for treating infections caused by the following microorganisms:

Aerobic Gram-positive Microorganisms: Including but not limited to Enterococcus faecalis, Enterococcus faecium, Staphylococcus aureus (including methicillin S. aureus), Staphylococcus epidermidis, Staphylococcus saprophyticus, Streptococcus pneumoniae, Streptococcus pyogenes, Staphylococcus haemolyticus, and Staphylococcus hominis;

Aerobic Gram-negative Microorganisms: Including but not limited to Campylobacter jejuni, Citrobacter diversus, Citrobacter freundii, Enterobacter cloacae, Escherichia coli, Haemophilus influenzae, Haemophilus parainfluenzae, Klebsiella pneumoniae, Moraxella catarrhalis, Morganella morganii, Neisseria gonorrhoeae, Proteus mirabilis, Proteus vulgaris, Providencia rettgeri, Providencia stuartii, Pseudomonas aeruginosa, Stenotrophomonas maltophila, Salmonella typhi, Serratia marcescens, Shigella boydii, Shigella dysenteriae, Shigella flexneri, Shigella sonnei, Acinetobacter Iwoffi, Aeromonas hydrophile, Edwardsiella tarda, Enterobacter aerogenes, Klebsiella oxytoca, Legionella pneumophila, Pasteurella multocida, Salmonella enteritidis, Vibrio cholerae, Vibrio parahaemolyticus, Vibrio vulnificus, Yersinia enterocolitica and H. Pylori.

Non-bacterial microorganisms: Mycoplasma, Legionella and Chlamydia.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient.

Frequency of dosage may also vary depending on the compound used and the particular disease treated. However, for treatment of most infectious disorders, a dosage regimen of 4 times daily or less is preferred and a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### Combination Administration

The compounds of the invention may also be useful in combination with other pharmaceutically active agents such as antibacterial agents, antiviral agents, antifungal agents, anti-inflammatories, interferon, efflux-pump inhibitors, and beta-lactamase inhibitors. Antibiotic agents include any molecule that tends to prevent, inhibit or destroy life and as such, includes anti-bacterial agents, anti-fungicides, anti-viral agents, and anti-parasitic agents.

Pharmaceutical compositions of the invention include single dosage forms containing of a compound of Formula I and one or more other active agent, dosage forms containing more than one compound of Formula I, and separate administration of a compound of Formula I with another active agent.

The following active agents, which are useful in combinations of the invention, may be isolated from an organism that produces the agent or synthesized by methods known to those of ordinary skill in the art of medicinal chemistry or purchased from a commercial source.

Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones (see Table below). Examples of antibiotic agents include, but are not limited to, Penicillin G (CAS Registry No.: 61-33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.: 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefmetazole (CAS Registry No.: 56796-20-4); Cefotetan (CAS Registry No.: 69712-56-7); Cefprozil (CAS Registry No.: 92665-29-7); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives.

Anti-fungals agents include but are not limited to Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, PyrroInitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconawle, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

Antiviral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, ZaIcitabine, Zidovudine, Acemannan, Acetylleucine, Amantadine, Amidinomycin, Delavirdine, Foscarnet, Indinavir, Interferon-α, Interferon-β, Interferon-γ, Kethoxal, Lysozyme, Methisazone, Moroxydine, Nevirapine, Podophyllotoxin, Ribavirin, Rimantadine, Ritonavir, Saquinavir, Stailimycin, Statolon, Tromantadine, and Xenazoic Acid.

Anti-inflammatory agents include, but are not limited to, Enfenamic Acid, Etofenamate, Flufenamic Acid, Isonixin, Meclofenamic Acid, Mefenamic Acid, Niflumic Acid, Talniflumate, Terofenamate, Tolfenamic Acid, Aceclofenac, Acemetacin, Alclofenac, Amfenac, Amtolmetin Guacil, Bromfenac, Bufexamac, Cinmetacin, Clopirac, Diclofenac, Etodolac, Felbinac, Fenclozic Acid, Fentiazac, Glucametacin, Ibufenac, Indomethacin, Isofezolac, Isoxepac, Lonazolac, Metiazinic Acid, Mofezolac, Oxametacine, Pirazolac, Proglumetacin, Sulindac, Tiaramide, Tolmetin, Tropesin, Zomepirac, Bumadizon, Butibufen, Fenbufen, Xenbucin, Clidanac, Ketorolac, Tinoridine, Alminoprofen, Benoxaprofen, Bermoprofen, Bucloxic Acid, Carprofen, Fenoprofen, Flunoxaprofen, Flurbiprofen, Ibuprofen, Ibuproxam, Indoprofen, Ketoprofen, Loxoprofen, Naproxen, Oxaprozin, Piketoprofen, Pirprofen, Pranoprofen, Protizinic Acid, Suprofen, Tiaprofenic Acid, Ximoprofen, Zaltoprofen, Difenamizole, Epirizole, Apazone, Benzpiperylon, Feprazone, Mofebutazone, Morazone, Oxyphenbutazone, Phenylbutazone, Pipebuzone, Propyphenazone, Ramifenazone, Suxibuzone, Thiazolinobutazone, Acetaminosalol, Aspirin, Benorylate, Bromosaligenin, Calcium Acetylsalicylate, Diflunisal, Etersalate, Fendosal, Gentisic Acid, Glycol Salicylate, Imidazole Salicylate, Lysine Acetylsalicylate, Mesalamine, Morpholine Salicylate, I-Naphthyl Salicylate, Olsalazine, Parsalmide, Phenyl Acetylsalicylate, Phenyl Salicylate, Salacetamide, Salicylamide O-Acetic Acid, Salicylsulfuric Acid, Salsalate, Sulfasalazine, Ampiroxicam, Droxicam, Isoxicam, Lornoxicam, Piroxicam, Tenoxicam, epsilon-Acetamidocaproic Acid, S-Adenosylmethionine, 3-Amino-4-hydroxylbutyric Acid, Amixetrine, Bendazac, Benzydamine, alpha-Bisabolol, Bucolome, Difenpiramide, Ditazol, Emorfazone, Fepradinol, Guaiazulene, Nabumetone, Nimesulide, Oxaceprol, Paranyline, Perisoxal, Proquazone, Superoxide Dismutase, Tenidap, Zileuton, 21-Acetoxypregnenolone, Alclometasone, Algestone, Amcinonide, Beclomethasone, Betamethasone, Budesonide, Chloroprednisone, Clobetasol, Clobetasone, Clocortolone, Cloprednol, Corticosterone, Cortisone, Cortivazol, Deflazacort, Desonide, Desoximetasone, Dexamethasone, Diflorasone, Diflucortolone, Difluprednate, Enoxolone, Fluazacort, Flucloronide, Flumethasone, Flunisolide, Fluocinolone Acetonide, Fluocinonide, Fluocortin Butyl, Fluocortolone, Fluorometholone, Fluperolone Acetate, Fluprednidene Acetate, Fluprednisolone, Flurandrenolide, Fluticasone Propionate, Formocortal, Halcinonide, Halobetasol Propionate, Halometasone, Halopredone Acetale, Hydrocortamate, Hydrocortisone, Loteprednol Etabonale, Mazipredone, Medrysone, Meprednisone, Methylprednisolone, Mometasone Furoate, Paramethasone, Prednicarbate, Prednisolone, Prednisolone 25-Diethylamino-acetate, Prednisolone Sodium Phosphate, Prednisone, Prednival, Prednylidene, Rimexolone, Tixocortol, Triamcinolone, Triamcinolone Acetonide, Triamcinolone Benetonide, and Triamcinolone Hexacetonide.

Compounds of the invention may be combined with one or more Beta lactamase inhibitor when used in combination with a beta-lactam class antibiotic, such as penicillin or cephalosporins. Beta-lactamase inhibitors include, but are not limited to Clavulanic acid, Sulbactam, Sultamacillin, and Tazobactam.

Compounds of the invention may also be combined with one or more efflux pump inhibitor, such as a quinazolinone efflux pump inhibitors, d-ornithine-d-homophenylalanine-3-aminoquinoline, Phe-Arg-b-naphthylamide, propafenone, a phenothiazine or thioxanthene efflux pump inhibitor, 1-aza-9-oxafluorenes, N-[4-[2-(3,4-dihydro-6,7-dimethoxy-2(1H)-isoquinolinyl)ethyl]phenyl]-9,10-dihydro-5-methoxy-9-oxo-4-Acridinecarboxamide, reserpine, Milbemycin, Cinchonine, Verapamil, L-phenylalanyl-N-2-naphthalenyl-L-Argininamide (and analogs), 5′-methoxyhydnocarpin-D, methylxanthines, FK506, a cyclosporine efflux pump inhibitor, Nocardamine and other siderophores, Amiodarone, Cyclosporin A, Ro11-2933 (DMDP), Quinidine, and the optical isomers of Propranolol, Quinine (SQ1) and Quinidine, Quinine-10,11-epoxide, Quercetin, Amitriptyline, Taxuspine C derivatives, Emodin, MC-002434; Agosterol A; Pheophorbide; pyridoquinolines such as 2,2′-[(2,8,10-trimethylpyrido[3,2-g]quinoline-4,6-diyl)bis(oxy)]bis[N,N-dimethyl-ethanamine, Gitonavir, and Gemfibrozil.

### Synthesis of Compounds

The compounds of the invention are prepared according to methods well-known to those skilled in the art of organic chemical synthesis. The starting materials used in preparing the compounds of the invention are known, made by known methods, or are commercially available.

It is recognized that the skilled artisan in the art of organic chemistry can readily carry out standard manipulations of organic compounds without further direction. Examples of such manipulations are discussed in standard texts such as J. March, Advanced Organic Chemistry, John Wiley & Sons, 1992.

The skilled artisan will readily appreciate that certain reactions are best carried out when other functionalities are masked or protected in the compound, thus increasing the yield of the reaction and/or avoiding any undesirable side reactions. Often, the skilled artisan utilizes protecting groups to accomplish such increased yields or to avoid the undesired reactions. These reactions are found in the literature and are also well within the scope of the skilled artisan. Examples of many such manipulations can be found in, for example, T. Greene, Protecting Groups in Organic Synthesis, John Wiley & Sons, 1981.

In addition, it is recognized that one optical isomer, including a diastereomer and enantiomer, or a stereoisomer, may have favorable properties over the other. When a racemic mixture is discussed herein, it is clearly contemplated to include both optical isomers, including diastereomers and enantiomers, or one stereoisomer substantially free of the other.

This invention is further illustrated by the following examples that should not be construed as limiting.

### EXAMPLES

The following abbreviations are used in the reaction schemes and synthetic examples, which follow. This list in not meant to be an all-inclusive list of abbreviations used in the application as additional standard abbreviations, which are readily understood by those skilled in the art of organic synthesis, may also be used in the synthetic schemes and examples.
Aq.—Aqueous
DIEA—N,N-Disopropylethylamine
DMA—Dimethylacetamide
DMF—N,N-Dimethylformamide
DMSO—Dimethyl sulfoxide
EtOAc—ethyl acetate
rt—Room Temperature
TFA—Trifluoroacetic acid
THF—Tetrahydrofuran

### General Methods

All nonaqueous reactions are performed under an atmosphere of dry argon gas using oven-dried glassware. The progress of reactions is monitored using liquid chromatography (UPLC) and/or thin-layer chromatography (TLC) on glass plates coated with Merck silica gel 60 (F₂₅₄). Flash column chromatography is performed on Merck silica gel 60 (230-400 mesh). The purity of target compounds and the progress of reactions are determined using the following liquid chromatographic method: 3.25-min gradient elution of increasing concentrations of acetonitrile in water (10-90%) containing 0.05% formic acid with a flow rate of 1.0 mL/min and UV (PDA) and mass detection on a Acquity UPLC BEH C18 150×2.1 mm 1.7 μm column. Unless noted otherwise, NMR spectra are recorded at ambient temperature in DMSO-d₆ using a Bruker Avance 300 spectrometer (¹H at 300.1 MHz, ¹³C at 75.5 MHz, and ¹⁹F at 282.4 MHz). The chemical shifts for ¹H and ¹³C are reported in parts per million (δ) relative to external tetramethylsilane and are referenced to signals of residual protons in the deuterated solvent. The chemical shifts for ¹⁹F are reported in parts per million (6) relative to external fluorotrichloromethane. All ¹³C and ¹⁹F NMR spectra are broadband ¹H decoupled.

### Example 1

### Synthesis of Boc Protected Pyrrolidine Diamine

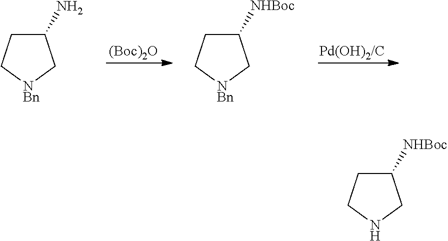 [SMILES: "(Boc)2O" ; N[C@H]1CCN(Cc2ccccc2)C1 ; CC(C)(C)OC(=O)N[C@H]1CCN(Cc2ccccc2)C1 ; "Pd(OH)2/C" ; CC(C)(C)OC(=O)N[C@H]1CCNC1]

from TCl

### Example 2

### Synthesis of (S)-2-Acetyl-7-(3-Aminopyrrolidin-1-Yl)-9-Cyclopropyl-6-Fluoro-3-Hydroxy-8-Methoxythieno[2,3-B]Quinolin-4(9H)-One

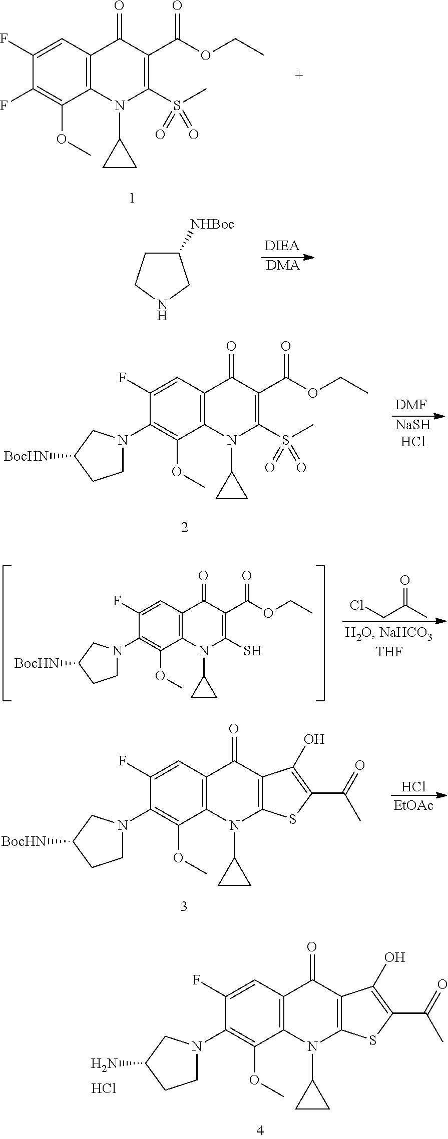

Step 1. Synthesis of (S)-ethyl 7-(3-((tert-butoxycarbonyl)amino)pyrrolidin-1-yl)-1-cyclopropyl-6-fluoro-8-methoxy-2-(methylsulfonyl)-4-oxo-1,4-dihydroquinoline-3-carboxylate (Compound 2)

The diamine (2.3 g, 1.5 equiv) is dissolved in N,N-dimethylacetamide (35 mL) and the solution is degassed with argon for 5 min. Then sulfone (ref. Org. Proc. Res. Dev. 2007, 11, 389-398) (3.36 g, 1 equiv) and diisopropylethylamine (7.3 mL, 5 equiv) is added and the resulting solution again degassed with argon for 5 minutes. This reaction mixture is then heated at 70° C. for 18 h under argon. The reaction mixture is dropped slowly into cold 1N HCl/10% NaCl sol. The precipitate is collected and dissolved in CH₂Cl₂. Organic layer is separated and dried over Na₂SO₄ and evap. 2.93 g of 2 as an orange yellow solid is obtained.

### Step 2. Synthesis of (S)-tert-Butyl (1-(2-Acetyl-9-cyclopropyl-6-fluoro-3-hydroxy-8-methoxy-4-oxo-4,9-dihydrothieno[2,3-b]quinolin-7-yl)pyrrolidin-3-yl)carbamate (Compound 3)

The sulfone 2 (2.9 g) is dissolved in DMF (30 mL) and argon is bubbled through the solution. Then solid sodium hydrosulfide (0.87 g, 3 equiv) is added while continuing to bubble argon. Then the reaction mixture is stirred at RT for 1 h under argon. The reaction mixture is dropped slowly into cold 1N HCl/10% NaCl sol. The precipitate is collected and dissolved in EtOAc and bubbled with Ar until Pb(OAc)₂ paper color no longer changes when spotted with the solution. The solvent is then removed.

The yellow solid is taken up in a 1:1 mixture of THF-water (60 mL). This solution is cooled in an ice bath and made basic with solid NaHCO₃ (pH˜9). Then 619 μL of chloro acetone (1.5 equiv) is added and the reaction mixture is stirred at RT till all the thiol is consumed and the intermediate has disappeared (about 2-3 h). The reaction mixture is dropped slowly into cold 1N HCl/10% NaCl sol. The precipitate is collected and washed with water and dried. (2.7 g). 0.5 g of this solid was purified by prep HPLC to give 0.2 g of the pure cyclized product 3.

### Step 3. Synthesis of the final product, (S)-2-Acetyl-7-(3-aminopyrrolidin-1-yl)-9-cyclopropyl-6-fluoro-3-hydroxy-8-methoxythieno[2,3-b]quinolin-4(9H)-one Hydrochloride (4)

The Boc-protected 3 (0.2 g) is taken up in EtOAc (8 mL) and 5 drops of conc. HCl are added. The solution is stirred at RT for 30 min. The precipitated solid is isolated by centrifugation. Then the solid is washed with EtOAc and dried under high vacuum to yield 0.150 g of final product (4).

### Example 3

### Synthesis and Testing of Additional Compounds

The preparation of compounds listed in Table I can be carried out under conditions analogous to those described in the examples above. Those of skill in the art will recognize routine changes in reaction conditions and starting materials needed to produce the listed compounds. For example, compound 5 in Table I, is prepared by the method shown above, except tert-butyl (1-(pyrrolidin-3-yl)ethyl)carbamate, prepared by the method shown in Schoeder, et al., J. Heterocyclic Chem., 29: 1481-1498 (1992), is used as the amine starting material. Compound activity was evaluated in a standard minimal inhibitory concentration assay such as the assay provided in Example 4. Compound cytotoxicity is determined using a standard assay of cytotoxicity such as the assay provided in Example 5. Compounds 10, 14, and 20 of Table I are provided as comparative examples.

| | | | HPLC | | | Cyto- |
|---|---|---|---|---|---|---|
| Cmp | | | t_{R} | | | toxicity |
| # | Structure | Name/NMR Data | (min) | MSSA | MRSA | Hep2 |
| 5 | 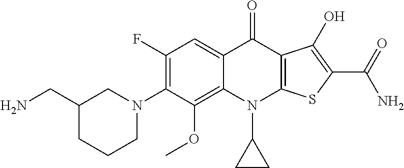 [SMILES: COc1c(N2CCCC(CN)C2)c(F)cc2c(=O)c3c(O)c(C(N)=O)sc3n(C3CC3)c12] | 7-(3-(aminomethyl)piperidin-1- yl)-9-cyclopropyl-6-fluoro-3- hydroxy-8-methoxy-4-oxo-4,9- dihydrothieno[2,3-b]quinoline- 2-carboxamide | | 0.5 | 2.00 | 30 |
| 6 | 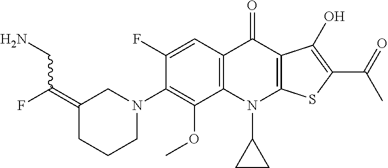 [SMILES: COc1c(N2CCCC(=C(F)CN)C2)c(F)cc2c(=O)c3c(O)c(C(C)=O)sc3n(C3CC3)c12] | 2-acetyl-7-(3-(2-amino-1- fluoroethylidene)piperidin-1- yl)-9-cyclopropyl-6-fluoro-3- hydroxy-8-methoxythieno [2,3-b]quinolin-4(9H)-one | | 0.25 | 0.50 | |
| 7 | 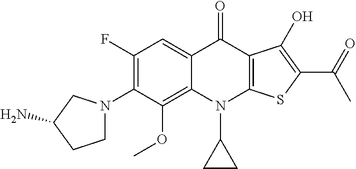 [SMILES: COc1c(N2CC[C@H](N)C2)c(F)cc2c(=O)c3c(O)c(C(C)=O)sc3n(C3CC3)c12] | (S)-2-acetyl-7-(3- aminopyrrolidin-1-yl)-9- cyclopropyl-6-fluoro-3- hydroxy-8-methoxythieno [2,3-b]quinolin-4(9H)-one ¹H (DMSO-d₆): δ 0.93 (m, 2H), 1.21 (m, 2H), 2.04 (m, 1H), 2.28 (m, 1H), 2.50 (s, 3H), 3.54 (s, 3H), 3.62 (m, 1H), 3.70-3.97 (m, 5H), 7.61 (d, J_{H-F} = 14.0 Hz, 1H), 8.17 (br, 3H). ¹⁹F (DMSO- d₆): δ −124.3 (s). | 1.54 | 0.13 | 0.25 | 35 |
| 8 | 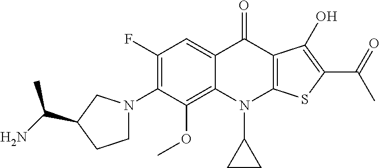 [SMILES: COc1c(N2CC[C@@H]([C@H](C)N)C2)c(F)cc2c(=O)c3c(O)c(C(C)=O)sc3n(C3CC3)c12] | 2-acetyl-7-((R)-3-((S)-1- aminoethyl)pyrrolidin-1- yl)-9-cyclopropyl-6-fluoro- 3-hydroxy-8-methoxythieno [2,3-b]quinolin-4(9H)-one ¹H (DMSO-d₆): δ 0.92 (m, 2H), 1.12-1.27 (m, 2H), 1.30 (d, J_{H-H} = 6.5 Hz, 3H), 1.74 (m, 1H), 2.09 (m, 1H), 2.41 (m, 1H), 2.48 (s, 3H), 3.29 (m, 1H), 3.51 (s, 3H), 3.57 (m, 1H), 3.64-3.90 (m, 4H), 7.57 (d, J_{H-F} = 14.0 Hz, 1H), 8.08 (br, 3H). ¹⁹F (DMSO-d₆): δ −124.2 (s). | 1.77 | 0.03 | 0.13 | 9 |
| 9 | 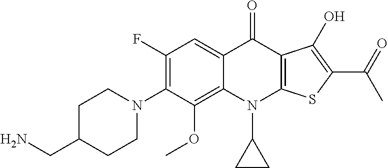 [SMILES: COc1c(N2CCC(CN)CC2)c(F)cc2c(=O)c3c(O)c(C(C)=O)sc3n(C3CC3)c12] | 2-acetyl-7-(4- (aminomethyl)piperidin-1- yl)-9-cyclopropyl-6- fluoro-3-hydroxy-8- methoxythieno[2,3-b] quinolin-4(9H)-one | 1.65 | 0.25 | 1 | 42 |
| 10 | 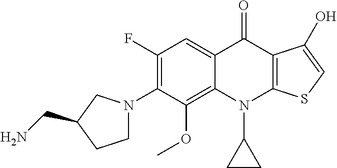 [SMILES: COc1c(N2CC[C@@H](CN)C2)c(F)cc2c(=O)c3c(O)csc3n(C3CC3)c12] | (S)-7-(3- (aminomethyl)pyrrolidin- 1-yl)-9-cyclopropyl-6- fluoro-3-hydroxy-8- methoxythieno[2,3-b] quinolin-4(9H)-one | 0.92 | 4 | | 24 |
| 11 | 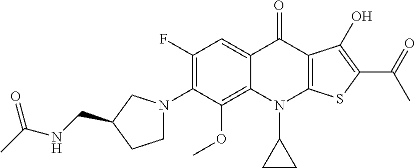 [SMILES: COc1c(N2CC[C@@H](CNC(C)=O)C2)c(F)cc2c(=O)c3c(O)c(C(C)=O)sc3n(C3CC3)c12] | (S)-N-((1-(2-acetyl-9- cyclopropyl-6-fluoro-3- hydroxy-8-methoxy-4- oxo-4,9-dihydrothieno [2,3-b]quinolin-7-yl) pyrrolidin-3-yl)methyl) acetamide | 2.22 | 0.13 | 0.5 | 60 |
| 12 | 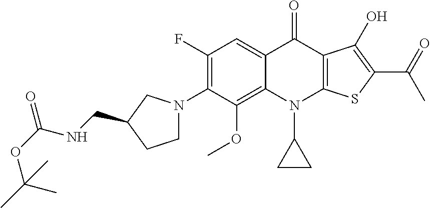 [SMILES: COc1c(N2CC[C@@H](CNC(=O)OC(C)(C)C)C2)c(F)cc2c(=O)c3c(O)c(C(C)=O)sc3n(C3CC3)c12] | (S)-2-acetyl-7-(3-(((tert- butoxycarbonyl)amino) methyl)pyrrolidin-1-yl)- 9-cyclopropyl-6- fluoro-8-methoxy-4- oxo-4,9-dihydrothieno [2,3-b]quinolin-3-yl 5-fluoro-2- methylbenzenesulfonate | 3.01 | 4 | >64 | |
| 13 | 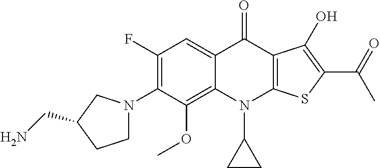 [SMILES: COc1c(N2CC[C@H](CN)C2)c(F)cc2c(=O)c3c(O)c(C(C)=O)sc3n(C3CC3)c12] | (R)-2-acetyl-7-(3- (aminomethyl)pyrrolidin- 1-yl)-9-cyclopropyl-6- fluoro-3-hydroxy-8- methoxythieno[2,3-b] quinolin-4(9H)-one | 1.58 | 0.13 | 0.25 | |
| 14 | 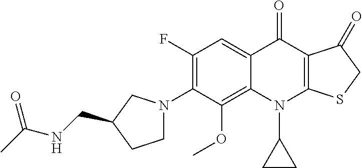 [SMILES: COc1c(N2CC[C@@H](CNC(C)=O)C2)c(F)cc2c(=O)c3c(n(C4CC4)c12)SCC3=O] | (S)-N-((1-(9-cyclopropyl- 6-fluoro-8-methoxy-3,4- dioxo-2,3,4,9-tetrahydrothieno [2,3-b]quinolin-7-yl) pyrrolidin-3-yl)methyl) acetamide | 1.37 | 32 | >64 | |
| 15 | 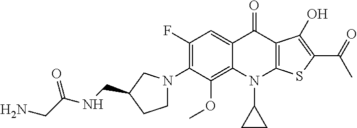 [SMILES: COc1c(N2CC[C@@H](CNC(=O)CN)C2)c(F)cc2c(=O)c3c(O)c(C(C)=O)sc3n(C3CC3)c12] | (S)-N-((1-(2-acetyl-9- cyclopropyl-6-fluoro-3- hydroxy-8-methoxy-4- oxo-4,9-dihydrothieno [2,3-b]quinolin-7-yl) pyrrolidin-3-yl)methyl)- 2-aminoacetamide | 1.51 | 0.5 | 1 | |
| 16 | 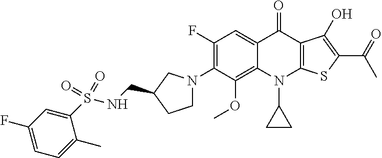 [SMILES: COc1c(N2CC[C@@H](CNS(=O)(=O)c3cc(F)ccc3C)C2)c(F)cc2c(=O)c3c(O)c(C(C)=O)sc3n(C3CC3)c12] | (R)-N-((1-(2-acetyl-9- cyclopropyl-6-fluoro-3- hydroxy-8-methoxy-4- oxo-4,9-dihydrothieno [2,3-b]quinolin-7-yl) pyrrolidin-3-yl) methyl)-5-fluoro-2- methylbenzenesulfonamide | 2.87 | 32 | >64 | |
| 17 | 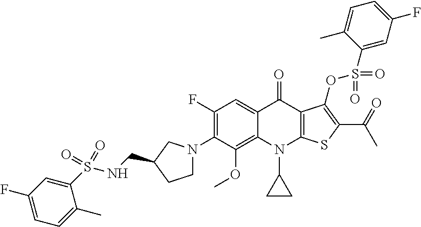 [SMILES: COc1c(N2CC[C@@H](CNS(=O)(=O)c3cc(F)ccc3C)C2)c(F)cc2c(=O)c3c(OS(=O)(=O)c4cc(F)ccc4C)c(C(C)=O)sc3n(C3CC3)c12] | (R)-2-acetyl--cyclopropyl- 6-fluoro-7-(3-((5-fluoro- 2-methylphenylsulfonamido) methyl)pyrrolidin-1-yl)-8- methoxy-4-oxo-4,9- dihydrothieno[2,3-b] quinolin-3-yl 5-fluoro-2- methylbenzenesulfonate | 3.14 | >64 | >64 | |
| 18 | 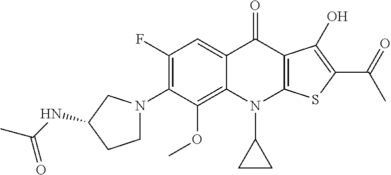 [SMILES: COc1c(N2CC[C@H](NC(C)=O)C2)c(F)cc2c(=O)c3c(O)c(C(C)=O)sc3n(C3CC3)c12] | (S)-N-(1-(2-acetyl-9- cyclopropyl-6-fluoro-3- hydroxy-8-methoxy-4- oxo-4,9-dihydrothieno [2,3-b]quinolin-7-yl) pyrrolidin-3-yl)acetamide | 1.94 | 0.25 | 1 | |
| 19 | 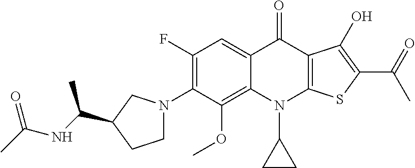 [SMILES: COc1c(N2CC[C@@H]([C@H](C)NC(C)=O)C2)c(F)cc2c(=O)c3c(O)c(C(C)=O)sc3n(C3CC3)c12] | N-((S)-1-((R)-1-(2- acetyl-9-cyclopropyl-6- fluoro-3-hydroxy-8- methoxy-4-oxo-4,9- dihydrothieno[2,3-b] quinolin-7-yl) pyrrolidin-3-yl)ethyl) acetamide | 2.28 | 0.25 | 0.5 | |
| 20 | 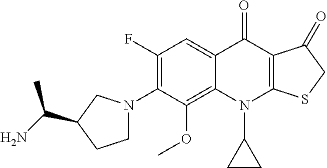 [SMILES: COc1c(N2CC[C@@H]([C@H](C)N)C2)c(F)cc2c(=O)c3c(n(C4CC4)c12)SCC3=O] | 7-((R)-3-((S)-1- aminoethyl)pyrrolidin- 1-yl)-9-cyclopropyl- 6-fluoro-8- methoxythieno[2,3-b] quinoline-3,4(2H,9H)- dione | 1.21 | 4 | 16 | |
| 21 | 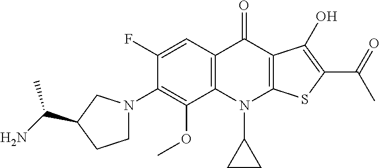 [SMILES: COc1c(N2CC[C@@H]([C@@H](C)N)C2)c(F)cc2c(=O)c3c(O)c(C(C)=O)sc3n(C3CC3)c12] | 2-acetyl-7-((R)-3-((R)-1- aminoethyl)pyrrolidin-1- yl)-9-cyclopropyl-6-fluoro- 3-hydroxy-8- methoxythieno[2,3-b] quinolin-4(9H)-one | 1.55 | 0.13 | 1 | |
| 22 | 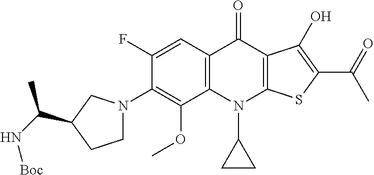 [SMILES: COc1c(N2CC[C@@H]([C@H](C)NC(=O)OC(C)(C)C)C2)c(F)cc2c(=O)c3c(O)c(C(C)=O)sc3n(C3CC3)c12] | tert-butyl ((S)-1-((R)-1- (2-acetyl-9-cyclopropyl- 6-fluoro-3-hydroxy-8- methoxy-4-oxo-4,9- dihydrothieno[2,3-b] quinolin-7-yl) pyrrolidin-3-yl) ethyl)carbamate | 3.01 | >64 | >64 | |
| 23 | 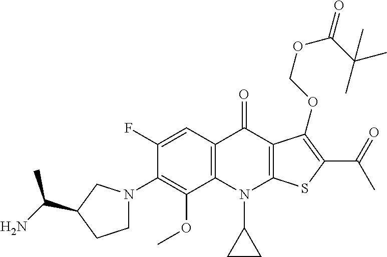 [SMILES: COc1c(N2CC[C@@H]([C@H](C)N)C2)c(F)cc2c(=O)c3c(OCOC(=O)C(C)(C)C)c(C(C)=O)sc3n(C3CC3)c12] | ((2-acetyl-7-((R)-3-((S)-1- aminoethyl)pyrrolidin-1- yl)-9-cyclopropyl-6- fluoro-8-methoxy- 4-oxo-4,9-dihydrothieno [2,3-b]quinolin-3-yl)oxy) methyl pivalate | 1.97 | | | |
| 24 | 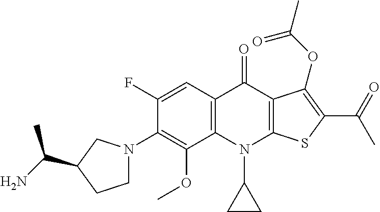 [SMILES: COc1c(N2CC[C@@H]([C@H](C)N)C2)c(F)cc2c(=O)c3c(OC(C)=O)c(C(C)=O)sc3n(C3CC3)c12] | 2-acetyl-7-((R)-3-((S)-1- aminoethyl)pyrrolidin-1- yl)-9-cyclopropyl-6- fluoro-8-methoxy- 4-oxo-4,9-dihydrothieno [2,3-b]quinolin- 3-yl acetate | 1.26 | | | |

Note: MSSA = S. aureus ATCC 29213; MRSA = fluoroquinolone-resistant S. aureus ATCC 700699; MIC in μg/mL

Compounds 10, 14, and 20 in Table I exist as keto-enol tautomers, i.e.:

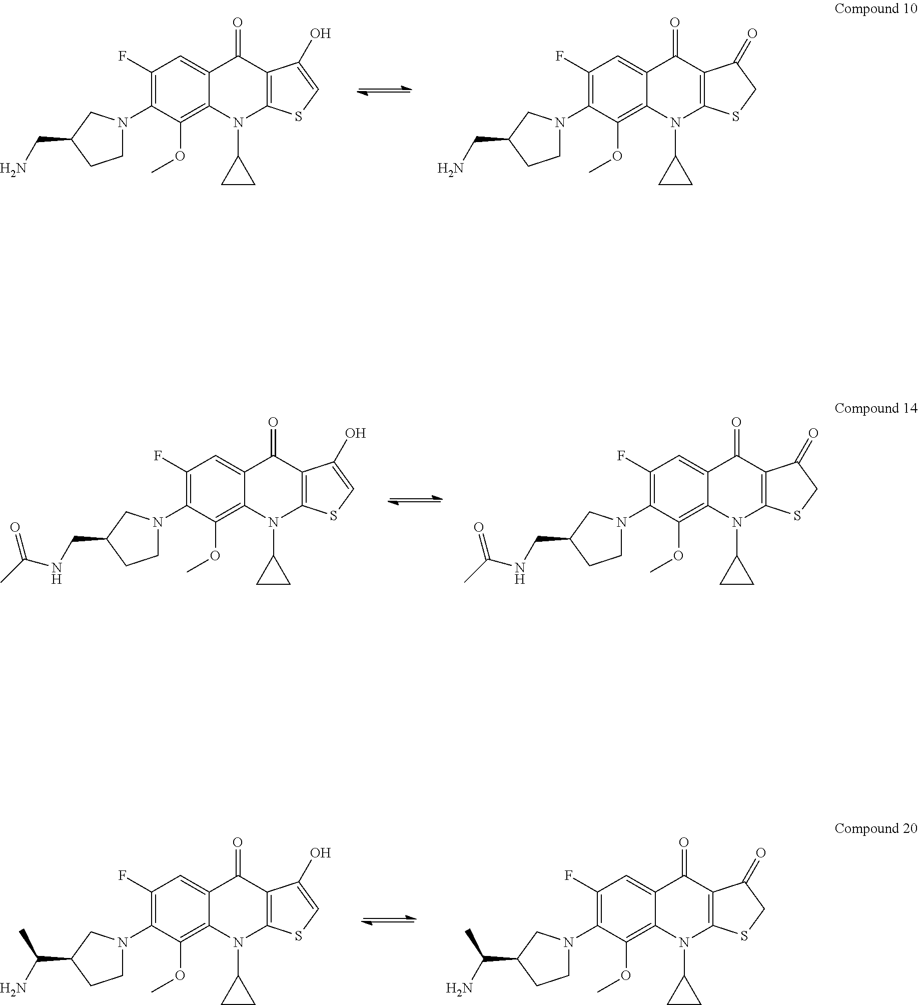 [SMILES: COc1c(N2CC[C@@H](CN)C2)c(F)cc2c(=O)c3c(O)csc3n(C3CC3)c12 ; COc1c(N2CC[C@@H](CN)C2)c(F)cc2c(=O)c3c(n(C4CC4)c12)SCC3=O ; COc1c(N2CC[C@@H](CNC(C)=O)C2)c(F)cc2c(=O)c3c(O)csc3n(C3CC3)c12 ; COc1c(N2CC[C@@H](CNC(C)=O)C2)c(F)cc2c(=O)c3c(n(C4CC4)c12)SCC3=O ; COc1c(N2CC[C@@H]([C@H](C)N)C2)c(F)cc2c(=O)c3c(O)csc3n(C3CC3)c12 ; COc1c(N2CC[C@@H]([C@H](C)N)C2)c(F)cc2c(=O)c3c(n(C4CC4)c12)SCC3=O]

### Example 4

### Antimicrobial Activity of Compounds—Minimum Inhibitory Concentration (MIC) Assay

The antimicrobial activity of the compounds of the invention may be evaluated by a number of methods, including the following visual minimum inhibitory concentration (MIC) assay. This assay determines the minimum concentration of compound required to inhibit growth of a bacterial strain.

Whole-cell antibacterial activity is determined by broth microdilution using conditions recommended by the NCCLS (see National Committee for Clinical Laboratory Standards. 2001. Performance standards for antimicrobial susceptibility testing: 11^{th} informational supplement. Vol. 21, no. 1, M100-S11. National Committee for Clinical Laboratory Standards, Wayne, Pa.). Test compounds are dissolved in DMSO and diluted 1:50 in Mueller-Hinton II broth (Becton-Dickinson) to produce a 256 μg/ml stock solution. In a 96-well microtiter plate, the compound solution is serially two-fold diluted in Mueller-Hinton II broth. After the compounds are diluted, a 50 μl aliquot of the test organism (˜1×10⁶ cfu/mL) is added to each well of the microtiter plate. The final test concentrations range from 0.125-128 μg/mL. Inoculated plates are incubated in ambient air at 37° C. for 18 to 24 hours. The organisms selected for testing included laboratory strains S. aureus ATCC 29213 and E. coli ATCC 25922 (strains purchased from American Type Culture Collection, Manassas, Va.) m, S. aureus FQR700699, and Paeruginosa 27853. The minimum inhibitory concentration (MIC) is determined as the lowest concentration of compound that inhibited visible growth of the test organism.

### Example 5

### Cell Viability Staining with Alamar Blue

To determine whether the microcidal effect observed against S. aureus and E. coli is specific to bacterial cells, compounds are screened for cell viability effects on several human cell types.

Optimal cell density is first determined by plating cells in a 96-well plate standard sterile tissue culture plates in 100 μl media, 10% FBS at six cell densities from 500 cells/well to 15,000 cells/well. A cell free well containing only media is used as a control. Cells are incubated at 37° C. in a 5% CO₂ incubator for 24 hours. 10% culture volume (10 ul) of Alamar Blue (Biosource, DAL1100, 100 mL) is then added. Cells are incubated at 37° C. in a 5% CO2 incubator and read in a Victor V plate reader, 544 nm excitation, 590 nm emission, at 3, 4, and 24 hours after the addition of Alamar Blue. The cell number vs. change in fluorescence is plotted to determine linearity of signal vs. cell number. The optimal density varies between 500-15,000 cells/well depending on the specific cell type. The optimal density is selected based on the highest number of cells that is still in the linear response range.

### Determination of Compound Cytotoxicity

Cells are plated at optimal cell density in a standard sterile tissue culture 96 well plate, and incubated at 37° C., O/N in a 5% CO₂ incubator. 12 to 48 hours post-plating media is removed. The cells are washed 1 or 2 times with 1×PBS and replaced with fresh media containing the test compound in 1% DMSO. 24 to 72 hours after addition of compound, the media is removed, and the cells washed 1 to 2 times with 1×PBS. Fresh media containing 1/10 volume of Alamar Blue is then added. Plates are incubated 4 hours at 37° C. in a 5% CO₂ incubator and read in a Victor V plate reader, 544 nm excitation, 590 nm emission.

Compounds are diluted to 20 micromolar in 1% DMSO and media and screened in duplicate to obtain single concentration cytotoxicity data. Eight concentration points from 0.78 micromolar to 100 micromolar, run in duplicate, are used to determine cytotoxicity CC50 values. Cells with 1% DMSO and media are used as a negative control, compounds having a known CC50 against a particular cell type are used as positive controls.

The change in fluorescence vs. concentration of test compound is plotted to determine the cytotoxicity of the compound.

What is claimed is:

1. A compound of the formula 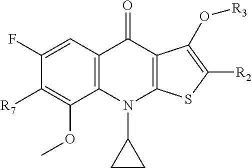
or a pharmaceutically acceptable salt thereof, wherein:
R₂ is amino, halogen, or —CORₐ, where Rₐ is C₁-C₆alkyl;
R₃ is hydrogen, C₁-C₆alkyl, C₁-C₆alkanoyl, (C₁-C₆alkylester)C₁-C₄alkyl, or a benzenesulfonyl group, each of which R₃ other than hydrogen is optionally substituted;
R₇ is a nitrogen-linked 5- to 6-membered heterocycloalkyl group, having 0 or 1 additional ring heteroatoms selected from N, O, and S, which nitrogen-linked 5- to 6-membered heterocycloalkyl group is substituted with at least 1 substituent independently chosen from (a) or (b) and 0 or 1 or more substituents independently chosen from (c); wherein
(a) is amino, aminoC₁-C₂alkyl, and aminoC₂-C₃alkenyl;
(b) is (C₁-C₆alkylcarbamate)C₀-C₄alkyl, (C₁-C₆alkylcarboxamide)C₀-C₄alkyl, and (benzenesulfonylamino)C₀-C₄alkyl where each of (a) and (b) other than amino is substituted with 0 to 2 substituents independently chosen from halogen, amino, C₁-C₄alkyl, and C₁-C₄alkoxy; and
(c) is halogen, hydroxyl, amino, C₁-C₄alkyl, C₂-C₄alkenyl, C₁-C₄alkoxy, C₁-C₂haloalkyl, and C₁-C₂haloalkoxy; with the proviso that the compound is not 9-cyclopropyl-6-fluoro-3-hydroxyl-8-methoxy-7-(piperazin-1-yl)-2-pivaloylthieno[2,3-b]quinolin-4(9H)-one; 2-acetyl-9-cyclopropyl-6-fluoro-3-hydroxyl-8-methoxy-7-(piperazin-1-yl)thieno[2,3-b]quinolin-4(9H)-one; (R)-2-acetyl-7-(3-(2-aminopropan-2-yl)pyrrolidin-1-yl)-9-cyclopropyl-6-fluoro-3-hydroxyl-8-methoxythieno[2,3-b]quinolin-4(9H)-one; (S)-2-acetyl-7-(3-(aminomethyl)pyrrolidin-1-yl)-9-cyclopropyl-6-fluoro-3-hydroxyl-8-methoxythieno[2,3-b]quinolin-4(9H)-one; (R)-7-(3-(2-aminopropan-2-yl)pyrrolidin-1-yl)-9-cyclopropyl-6-fluoro-3-hydroxyl-8-methoxy-2-propionylthieno[2,3-b]quinolin-4(9H)-one; 2-acetyl-7-(3-(aminomethyl)piperidin-1-yl)-9-cyclopropyl-6-fluoro-3-hydroxyl-8-methoxythieno[2,3-b]quinolin-4(9H)-one; or 2-acetyl-7-(3-(2-amino-1-fluoroethylidene)piperidin-1-yl)-9-cyclopropyl-6-fluoro-3-hydroxyl-8-methoxythieno[2,3-b]quinolin-4(9H)-one.

2. A compound or salt of claim 1 wherein,
R₂ is amino, halogen, or —CORₐ, where Rₐ is C₁-C₆alkyl.

3. A compound or salt of claim 2, wherein R₂ is acetyl.

4. A compound or salt of claim 1, wherein R₃ is hydrogen, or a benzenesulfonyl group substituted with 0 or 1 or more substituents independently chosen from halogen, hydroxyl, amino, C₁-C₄alkyl, C₂-C₄alkenyl, C₁-C₄alkoxy, C₁-C₂haloalkyl, and C₁-C₂haloalkoxy.

5. A compound or salt of claim 4, wherein R₃ is hydrogen.

6. A compound or salt of claim 4, wherein R₃ is a benzenesulfonyl group substituted with 0 or 1 or 2 substituents independently chosen from halogen, hydroxyl, amino, methyl, methoxy, trifluoromethyl, and trifluoromethoxy.

7. A compound or salt of claim 1, wherein R₃ is C₁-C₄alkanoyl or (C₁-C₆alkylester)C₁-C₂alkyl.

8. A compound or salt of claim 1 wherein,
R₇ is a nitrogen-linked 5- to 6-membered heterocycloalkyl group, having 0 or 1 additional ring heteroatoms selected from N, O, and S, which nitrogen-linked 5- to 6-membered heterocycloalkyl group is substituted with at least 1 substituent independently chosen from (a) or (b) and 0 or 1 or more substituents independently chosen from (c); wherein
(a) is amino, aminoC₁-C₂alkyl, and aminoC₁-C₃alkenyl;
(b) is (C₁-C₆alkylcarbamate)C₀-C₄alkyl, (C₁-C₆alkylcarboxamide)C₀-C₄alkyl, and (benzenesulfonylamino)C₀-C₄alkyl, where each of (a) and (b) is other than amino substituted with 0 to 2 substituents independently chosen from halogen, amino, C₁-C₄alkyl, and C₁-C₄alkoxy; and
(c) is halogen, hydroxyl, amino, C₁-C₄alkyl, C₂-C₄alkenyl, C₁-C₄alkoxy, C₁-C₂haloalkyl, and C₁-C₂haloalkoxy.

9. A compound or salt of claim 8, wherein R₇ is pyrrolidinyl or piperidinyl, each of which is substituted with at least 1 substituent independently chosen from (a) or (b) and 0 or 1 or 2 substituents chosen from (c).

10. A compound or salt of claim 9, wherein R₇ is pyrrolidinyl or piperidinyl, each of which is substituted with at least 1 substituent independently chosen from (a) or (b); wherein (a) is amino and aminoC₁-C₂alkyl; and (b) is (C₁-C₆alkylcarbamate)C₁-C₂alkyl, (C₁-C₂alkylcarboxamide)C₁-C₂alkyl, and (benzenesulfonylamino)C₁-C₂alkyl, where each of (b) is substituted with 0 to 2 substituents independently chosen from halogen, amino, and methyl.

11. A compound or salt of claim 1, wherein the compound is
2-acetyl-7-(3-(2-amino-1-fluoroethylidene)piperidin-1-yl)-9-cyclopropyl-6-fluoro-3-hydroxy-8-methoxythieno[2,3-b]quinolin-4(9H)-one;
(S)-2-acetyl-7-(3-aminopyrrolidin-1-yl)-9-cyclopropyl-6-fluoro-3-hydroxy-8-methoxythieno[2,3-b]quinolin-4(9H)-one;
2-acetyl-7-((R)-3-((S)-1-aminoethyl)pyrrolidin-1-yl)-9-cyclopropyl-6-fluoro-3-hydroxy-8-methoxythieno[2,3-b]quinolin-4(9H)-one;
2-acetyl-7-(4-(aminomethyl)piperidin-1-yl)-9-cyclopropyl-6-fluoro-3-hydroxy-8-methoxythieno[2,3-b]quinolin-4(9H)-one;
(S)—N-((1-(2-acetyl-9-cyclopropyl-6-fluoro-3-hydroxy-8-methoxy-4-oxo-4,9-dihydrothieno[2,3-b]quinolin-7-yl)pyrrolidin-3-yl)methyl)acetamide;
(S)-2-acetyl-7-(3-(((tert-butoxycarbonyl)amino)methyl)pyrrolidin-1-yl)-9-cyclopropyl-6-fluoro-8-methoxy-4-oxo-4,9-dihydrothieno[2,3-b]quinolin-3-yl 5-fluoro-2-methylbenzenesulfonate;
(R)-2-acetyl-7-(3-(aminomethyl)pyrrolidin-1-yl)-9-cyclopropyl-6-fluoro-3-hydroxy-8-methoxythieno[2,3-b]quinolin-4(9H)-one;
(S)—N-((1-(2-acetyl-9-cyclopropyl-6-fluoro-3-hydroxy-8-methoxy-4-oxo-4,9-dihydrothieno[2,3-b]quinolin-7-yl)pyrrolidin-3-yl)methyl)-2-aminoacetamide;
(R)—N-((1-(2-acetyl-9-cyclopropyl-6-fluoro-3-hydroxy-8-methoxy-4-oxo-4,9-dihydrothieno[2,3-b]quinolin-7-yl)pyrrolidin-3-yl)methyl)-5-fluoro-2-methylbenzenesulfonamide;
(R)-2-acetyl-9-cyclopropyl-6-fluoro-7-(3-((5-fluoro-2-methylphenylsulfonamido)methyl)pyrrolidin-1-yl)-8-methoxy-4-oxo-4,9-dihydrothieno[2,3-b]quinolin-3-yl 5-fluoro-2-methylbenzenesulfonate;
(S)—N-(1-(2-acetyl-9-cyclopropyl-6-fluoro-3-hydroxy-8-methoxy-4-oxo-4,9-dihydrothieno[2,3-b]quinolin-7-yl)pyrrolidin-3-yl)acetamide;
N—((S)-1-((R)-1-(2-acetyl-9-cyclopropyl-6-fluoro-3-hydroxy-8-methoxy-4-oxo-4,9-dihydrothieno[2,3-b]quinolin-7-yl)pyrrolidin-3-yl)ethyl)acetamide;
2-acetyl-7-((R)-3-((R)-1-aminoethyl)pyrrolidin-1-yl)-9-cyclopropyl-6-fluoro-3-hydroxy-8-methoxythieno[2,3-b]quinolin-4(9H)-one;
tert-butyl((S)-1-((R)-1-(2-acetyl-9-cyclopropyl-6-fluoro-3-hydroxy-8-methoxy-4-oxo-4,9-dihydrothieno[2,3-b]quinolin-7-yl)pyrrolidin-3-yl)ethyl)carbamate;
((2-acetyl-7-((R)-3-((S)-1-aminoethyl)pyrrolidin-1-yl)-9-cyclopropyl-6-fluoro-8-methoxy-4-oxo-4,9-dihydrothieno[2,3-b]quinolin-3-yl)oxy)methyl pivalate; or
2-acetyl-7-((R)-3-((S)-1-aminoethyl)pyrrolidin-1-yl)-9-cyclopropyl-6-fluoro-8-methoxy-4-oxo-4,9-dihydrothieno[2,3-b]quinolin-3-yl acetate.

12. A pharmaceutical composition comprising a compound or salt of claim 1 together with a pharmaceutically acceptable carrier.

13. A method of treating a bacterial infection in a patient comprising administering a therapeutically effective amount of compound or salt of claim 1 to the patient having a bacterial infection.

14. The method of claim 12, wherein the bacterial infection is a MRSA infection.
